(19) United States
(12) Patent Application Publication
(10) Pub. No.: US 2023/0310675 A1
(43) Pub. Date: Oct. 5, 2023

(54) Hygienic Interface for Electronic Devices

(71) Applicant: Judson Gudgel, Tulsa, OK (US)

(72) Inventor: Judson Gudgel, Tulsa, OK (US)

(21) Appl. No.: 18/022,484

(22) PCT Filed: Aug. 23, 2021
(86) PCT No.: PCT/US2021/047129

§ 371 Date: Feb. 21, 2023

Related U.S. Application Data
(60) Provisional application No. 63/068,674, filed on Aug. 21, 2020.

(51) Int. Cl. A61L 2/10; B67D 7/04; G06F 3/02

(52) U.S. Cl. CPC A61L 2/10; B67D 7/04; G06F 3/02; B67D 7/32

(57) ABSTRACT

Disclosed are hygienic interfaces for use with electronic devices employing keypad keys which incorporate UVC emitting LEDs. The interfaces work in conjunction with controller to control access to human touch surfaces. Access to those services are denied when they are not hygienic.

### TECHNICAL FIELD

The present invention relates to interfaces for electronic devices. The present invention particularly relates to hygienic interfaces.

### BACKGROUND

UVC lamps used for disinfection purposes may pose potential health and safety risks depending on the UVC wavelength, dose, and duration of radiation exposure. Like all light, the intensity of the impact of photons decreases as the square of the distance between the source and the target.

It would be desirable in the art of inputting data or commands to electronic devices, where those devices are in public locations, to ensure that those input devices are hygienic. It would also be desirable in the art if the input devices could be maintained in a hygienic state safely.

### SUMMARY

In one aspect, the invention is a hygienic interface for electronic devices, comprising: a keypad; at least one key operatively connected to the keypad, wherein the at least one key has a body defined by a contact surface and an opposing surface; and at least one diode positioned in the body and configured to emit a UV-C light, wherein the body is substantially transparent to the UV-C light.

In another aspect, the invention is hygienic interface for a computer comprising: an input component selected from the group consisting of a keyboard, a computer mouse, and combination thereof, at least one key operatively connected to the input component, wherein the at least one key has a body defined by a contact surface and an opposing surface; and at least one diode positioned in the body and configured to emit a UV-C light, wherein the body is substantially transparent to the UV-C light.

In still another aspect, the invention is a process for inputting data or commands to an electronic device comprising employing a hygienic interface to input data or commands to the electronic device.

### BRIEF DESCRIPTION OF THE DRAWINGS

FIG. 1 is an illustration of a keypad key showing a contact surface.

FIG. 2 is a side view of the keypad key illustrated in FIG. 1.

FIG. 3 is an illustration of a gas pump wherein the access to a keypad is blocked by the housing of an embodiment of the present invention.

FIG. 4 the same gas pump as is shown in FIG. 3 except that the housing is now open and the keypad is accessible.

FIG. 5 is a same gas pump as is illustrated in FIGS. 3 and 4 but additionally showing a second housing blocking access to the dispensing nozzle.

### DETAILED DESCRIPTION

Ultraviolet light of a particular range of wavelengths, intensities, and durations can kill or inhibit growth of microorganisms. Specifically, ultraviolet radiation in the range of 200 nanometer (nm) to 300 nm is effective against airborne and surface bacteria, viruses, yeasts, and molds. For most microorganisms, the peak inactivation wavelength is at or about 260 nm. Embodiments of the present invention employ light emitting diodes (LEDs) selected from those that produce UVC radiation ranging from about 200 to about 300 nm. Such LEDs can be selected from those that produce a very narrow peak wavelength and those which are able to produce broader wavelengths.

When targeted at a specific bacteria or virus, the UVC emitting LEDs may be selected from those having very narrow peak wavelength production in a targeted wavelength. For example, in some embodiments, it may be desirable to select LEDs that produce a peak emission at about 270 nm. In other embodiments it may be more desirable to select an LED that has a peak emission at about 250 nm. In still another embodiment multiple LEDs may be employed in the keys of the application for efficiency at killing multiple organisms.

One disadvantage of employing UVC radiation is the possibility that it could have adverse health effects for users. Advantageously, the embodiments of the present invention mitigate this in 2 ways. One way that the embodiments of the application mitigate UV exposure to users is by taking advantage of the effect of incorporating the LEDs into the body of the keys. Since power falls off at a rate of the square of the distance from the light source and the target, the keys of the present application are assured of having the maximum amount of radiation.

The second way in which the embodiments of the present invention mitigate UV radiation exposure for users is by shielding users from the UV radiation when the UVC emitting LEDs are activated. For the purposes of this application, the term shielding means employing a barrier which is opaque to ultraviolet light between all the UVC emitting LEDs and users of all embodiments of the present application.

In one embodiment, the invention is a hygienic interface for electronic devices, comprising: a keypad; at least one key operatively connected to the keypad, wherein the at least one key has a body defined by a contact surface and an opposing surface; and at least one diode positioned in the body and configured to emit a UV-C light, wherein the body is substantially transparent to the UV-C light. For the purposes of this application, the term hygienic means that any surfaces that would be touched by user has been treated with ultraviolet light for a time and at an intensity sufficient to eliminate or at least mitigate undesirable bacteria and viruses.

Turning now to FIG. 1, a top surface vies of a key of the keypad is shown (101). In most embodiments, the contact surface will typically have a letter, number, or symbol embossed upon or otherwise applied to it.

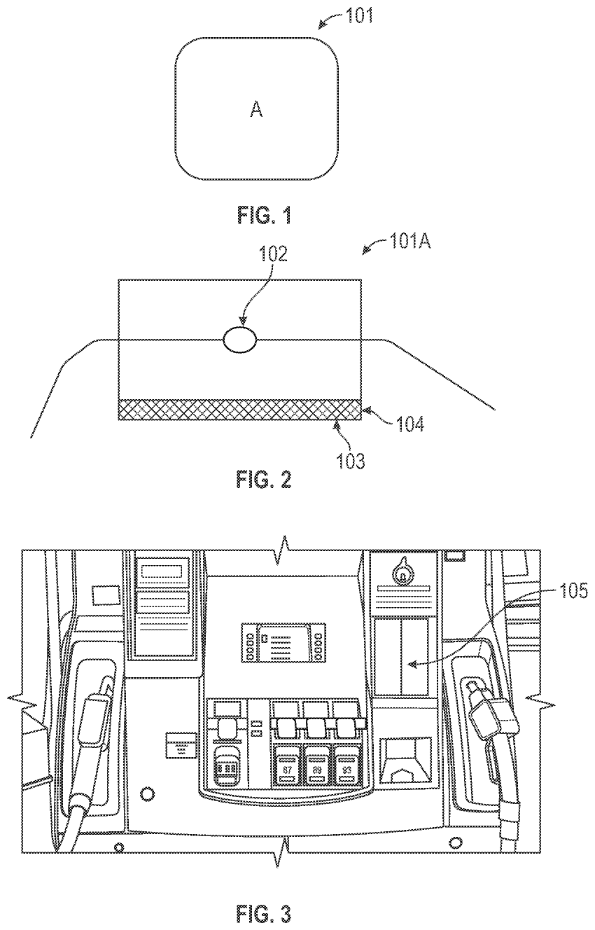

Turning now to FIG. 2, a side view of the key body is shown (101A). the body of the key can be solid or hollow but will contain at least one UVC emitting LED (102). The key will also be prepared employing either a glass or plastic composition which is substantially transparent to ultraviolet light. In some embodiments the body and nonreflective surfaces of the key will absorb less than 15% of the ultraviolet light emitted by the LED or LEDs. In other embodiments, the body and nonreflective services the key will absorb less than 10% of the ultraviolet light emitted by the LED or LEDs. And in still other embodiments, the body and nonreflective surfaces of the key will absorb less than 5% of the ultraviolet light emitted by the LED or LEDs.

Also shown in FIG. 2, the bottom (opposing side) (103) is either prepared with or has been treated with a material that is substantially reflective to ultraviolet light (104). Not shown but within the scope of the present application, the sides of the key are also reflective to ultraviolet light. In some embodiments it would be desirable that the maximum amount of ultraviolet light produced by the UVC emitting LEDs is directed up through the contact surface in order to minimize the period of time necessary to eliminate or at least mitigate undesirable organisms.

Turning now to FIG. 3, a gas pump is shown covering a keypad (105). In FIG. 3, the housing for the interface is in a closed position. When the housing is closed, the UV emitting LEDs in the keypad can be energized. In some embodiments the surface of the housing adjacent to the keypad may also have one or more LEDs to increase the amount of ultraviolet light treating the actual keys. The LEDs in the surface of the housing can also treat the remaining surface of the keypad to further mitigate the possibility of potentially infectious organisms being present.

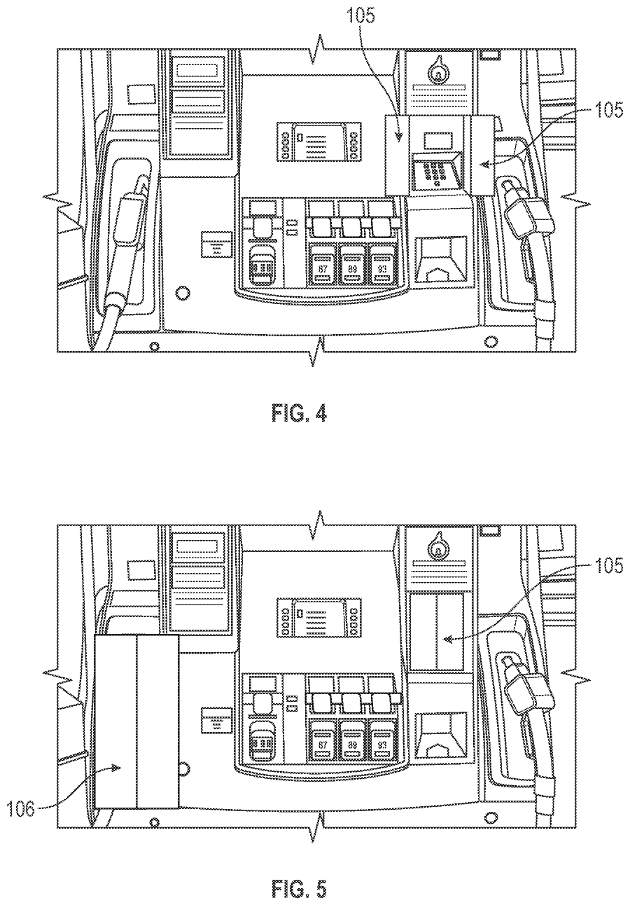

Turning now to FIG. 4, the keypad housing (105) is shown in the open position. When in the open position the keypad is accessible for use.

Finally, turning now to FIG. 5, in addition to the keypad being covered, by a first housing (105), the nozzle of the fuel pump is enclosed within a second housing (106). In this embodiment, all surfaces of the nozzle that are subject to skin contact are prepared employing a polymer or glass and have embedded therein as many UVC emitting LEDs as is necessary to render the nozzle hygienic.

Generally speaking, a single gas pump could conceivably employ as many as 4 or 5 or even more devices of the present application in order to ensure that all contact services on the pump have been rendered hygienic.

The hygienic interfaces of the application may have their own power source or may share a power source separate from that of the pump. In some embodiments the power source may be the same as that of the pump itself

Similarly, the controller for the hygienic interfaces may be the same as that which runs the fuel pump; or the hygienic interfaces may share or each individually have their own controller.

The controller referenced above can be configured to accept data from a motion detector. In one embodiment, it may be desirable to keep the housing closed except when the interfaces in use. In an alternative embodiment, the housing can be closed only during those periods where it is being treated with UVC radiation. Further, the controller can be used to actuate servos on all of the housings of the embodiments of the application.

In still another embodiment, the controller can be employed to change the duration of irradiation times based on known pathogens. For example, if a bacterial infection is in a pandemic stage, then the interfaces of the present application can be tuned to irradiate human contact services for time sufficient to eradicate that particular pathogen.

The end uses to which the hygienic interfaces of the present application can be applied are numerous. For example, a point-of-sale device is such a use. Other uses include an automated teller device, a payment terminal, a fuel pump, charging station, an entry control device, a digital lock, and combinations thereof. Other uses include entertainment and gambling devices such as a slot machine or video game.

As referenced earlier, the UVC emitting LEDs emit at a power sufficient to render hygienic the interface. Desirably, the LEDs will emit at a power level of at least 30 mW. In some embodiments they will emitted at power level of at least 33 mW.

In still another embodiment the present invention includes A hygienic interface for a computer comprising: an input component selected from the group consisting of a keyboard, a computer mouse, and combination thereof, at least one key operatively connected to the input component, wherein the at least one key has a body defined by a contact surface and an opposing surface; and at least one diode positioned in the body and configured to emit a UV-C light, wherein the body is substantially transparent to the UV-C light. In these embodiments, the interface is a keyboard, mouse, trackball, light pen, and combinations thereof

In addition to the interfaces themselves, the process of using those interfaces are also within the scope of the application. For example, in one embodiment a user will approach a device such as a gas pump employing interfaces of the application. A motion detector upon detecting the approach of a user will then actuate the interface. If the interface is already hygienic, then the access door to the housing will open granting the user access to the interface. Once a user has successfully negotiated the payment authorization process of the pump, then the interface will then actuate any other housings to grant access to a delivery nozzle.

In another embodiment, when the user approaches the interface and the interface is not yet hygienic, the interface may then display how much longer before a hygienic condition can be reached. In still another embodiment, the user may elect to override the delay between uses so that they can accept the risk of using an unhygienic interface.

While a gas pump is used for illustration, the method and apparatus of the present invention applies to any embodiment where the basic elements of the invention are present. Components such as stylus having a transparent body and an internal UV emitting LCD are also within the scope of the application.

What is claimed:

1. A hygienic interface for electronic devices, comprising:
a keypad;
at least one key operatively connected to the keypad, wherein the at least one key has a body defined by a contact surface and an opposing surface; and
at least one diode positioned in the body and configured to emit a UV-C light, wherein the body is substantially transparent to the UV-C light.

2. The hygienic interface of claim 1, wherein the body has a first zone located between the diode and the contact surface and a second zone located between the diode and the opposing surface, wherein the contact surface is substantially transparent to the UV-C light and the opposing surface is reflective to the UV-C light.

3. The hygienic interface of claim 2, wherein the contact surface and the opposing surface define a top and a bottom of the body, respectively, of the at least one key, and wherein the body is further defined by a plurality of sides that are configured to be reflective of the UV-C light.

4. The hygienic interface of claim 1, further comprising a power source and a controller configured to power and control the hygienic interface.

5. The hygienic interface of claim 4, further comprising a motion detector configured to detect motion of a body proximate to the keyboard.

6. The hygienic interface of claim 5, further comprising:
a first housing configured to enclose the keypad; and
at least one additional diode configured to emit UV-C light configured to irradiate at least a portion of the keypad when the first housing at least partially encloses the keypad.

7. The hygienic interface of claim 6, further comprising a first servo configured to open the first housing when the motion detector detects motion.

8. The hygienic interface of claim 7, wherein the keypad is configured to input information to a point of sale device.

9. The hygienic interface of claim 8 wherein the point of sale device is selected from the group comprising: an automated teller device, a payment terminal, a fuel pump, an entry control device, a digital lock, and combinations thereof

10. The hygienic interface of claim 9 wherein the point of sale device dispenses a product.

11. The hygienic interface of claim 10 wherein the point of sale device is a fuel pump which dispenses fuel employing a nozzle.

12. The hygienic interface of claim 11 wherein the nozzle is enclosed within a second housing.

13. The hygienic interface of claim 12 wherein the second housing is configured to be opened employing a second servo.

14. The hygienic interface of claim 13 wherein the second housing has additional UV-C light emitting diodes configured to irradiate at least part of the surface of the nozzle when the housing is closed.

15. The hygienic interface of claim 14 wherein the human contact points of the nozzle include a polymer having UV-C light emitting diodes embedded therein.

16. The hygienic interface of claim 15 wherein the UV-C light emitting diodes emit UV-C light at a power intensity sufficient to sanitize a surface.

17. The hygienic interface of claim 16 wherein the UV-C light emitting diodes emit UV-C light at a power intensity of at least 30 mW.

18. The hygienic interface of claim 17 wherein the UV-C light emitting diodes emit UV-C light at a power intensity of at least 33 mW.

19. The hygienic interface of claim 1 wherein the body absorbs less than 15% of the UV-C light.

20. The hygienic interface of claim 19 wherein the body absorbs less than 10% of the UV-C light.

21. The hygienic interface of claim 20 wherein the body absorbs less than 5% of the UV-C light.

22. The hygienic interface of claim 2 wherein the reflective surfaces reflect at least 50% of UV-C light.

23. A hygienic interface for a computer comprising:
An input component selected from the group consisting of a keyboard, a computer mouse, and combination thereof,
at least one key operatively connected to the input component, wherein the at least one key has a body defined by a contact surface and an opposing surface; and
at least one diode positioned in the body and configured to emit a UV-C light, wherein the body is substantially transparent to the UV-C light.

24. The hygienic interface of claim 23, wherein the body has a first zone located between the diode and the contact surface and a second zone located between the diode and the opposing surface, wherein the contact surface is substantial transparent to the UV-C light and the opposing surface is reflective to the UV-C light.

25. A process for inputting data or commands to an electronic device comprising employing a hygienic interface of claim 4 to input data or commands to the electronic device.

26. The process of claim 25 wherein the controller is preprogrammed to require a period of irradiation between uses and the period of irradiation is selected to be useful for eliminating or mitigating a specific bacteria or virus.
